(19) United States
(12) Patent Application Publication
(10) Pub. No.: US 2022/0079858 A1
(43) Pub. Date: Mar. 17, 2022

(54) Esteramine Compositions

(71) Applicant: Stepan Company, Northfield, IL (US)

(72) Inventors: James A. Faunce, North Aurora, IL (US); Renata A. Butikas, Chicago, IL (US); Sarah E. Kovach, Palatine, IL (US); Teresa Germain, Orland Park, IL (US); Patrick Shane Wolfe, Palatine, IL (US); Anatoliy A. Dameshek, Indian Creek, IL (US); Dennis S. Murphy, Libertyville, IL (US)

(21) Appl. No.: 17/519,900

(22) Filed: Nov. 5, 2021

Related U.S. Application Data
(63) Continuation of application No. PCT/US2020/031523, filed on May 5, 2020.
(60) Provisional application No. 62/844,072, filed on May 6, 2019.

(51) Int. Cl. A61K 8/41; A61K 8/37; A61Q 5/12; A61Q 19/00

(52) U.S. Cl. CPC A61K 8/416; A61Q 19/00; A61Q 5/12; A61K 8/375

(57) ABSTRACT

Compositions comprising at least one esteramine active are disclosed in which the esteramine provides conditioning, softening, and/or cleaning properties. The compositions are useful for hair care, as well as in other applications, such as cleaning compositions, fabric softening compositions, and skin care compositions. Also disclosed are esteramine compositions that comprise a synergistic mixture of at least one esteramine and mono- and di-glycerides that provides better wet and dry combing than either the esteramine or mono- and di-glycerides alone. The synergistic mixture comprises about 50% to about 90% by weight esteramines and about 10% to about 50% by weight mono- and diglycerides.

### CROSS-REFERENCE TO RELATED APPLICATIONS

This application is a continuation of and claims priority to PCT Application No. US2020/031523, filed May 5, 2020, which claims priority to U.S. Provisional Application No. 62/844,072, filed May 6, 2019. The entire specifications of the PCT and provisional application referred to above are hereby incorporated by reference.

### FIELD OF THE INVENTION

The present technology relates to compositions that comprise at least one esteramine compound as an active ingredient that provides conditioning, softening, and/or cleaning properties. The compositions are useful for hair care, as well as in other applications, such as cleaning compositions, fabric softening compositions, and skin care compositions. The present technology also relates to esteramine compositions that comprise a synergistic mixture of at least one esteramine and mono- and di-glycerides that provides better wet and dry combing than either the esteramine or mono- and di-glycerides alone.

### BACKGROUND OF THE INVENTION

Quaternary ammonium compounds have been used as hair conditioning agents for a number of years. Two of the most common hair conditioner agents are behentrimonium chloride (BTAC) and cetrimonium chloride (CETAC). Although both actives work well as conditioning agents, they have certain drawbacks. BTAC and CETAC have unfavorable environmental profiles and their use in conditioning compositions has been under scrutiny. Since 2014, the European Union has restricted these agents in both leave-on and rinse-off products. As raw materials, both BTAC and CETAC are irritating and/or toxic if in contact with skin, and are very toxic to aquatic life, with long-lasting effects. Another drawback is that liquid forms of BTAC and CETAC are only available at fairly low active concentrations of 25-30% active. Products with higher concentrations of actives are available, but typically only as pastilles or solids that require heating/melting or dilution with flammable solvents, such as isopropyl alcohol (IPA).

Manufacturers have sought other cationic compounds that can deliver conditioning performance without the drawbacks of BTAC and CETAC. Esterquat quaternary ammonium compounds (esterquats) have been used as a hair conditioning active. Such esterquats are typically made from fatty acids reacted with an amine, such as triethanolamine (TEA) or methyl diethanolamine (MDEA) and then quaternized. Use of fatty acids allows better control over the fatty acid chains reacting with the alkanolamine to make the esteramine, whose amine portion is then quaternized, and provides a “pure” molecule compared with oils containing fatty acids in triglyceride form. Although esterquats are less toxic than BTAC and CETAC from an environmental standpoint, they often do not perform as well as BTAC and CETAC as a hair conditioning agent. Esterquats are also usually in a solid or paste form, and require heating/melting or dilution with a solvent, such as IPA or ethanol, which releases volatile organic compounds (VOCs) into the environment.

Amidoamines are another class of compounds that can deliver hair conditioning performance. Amidoamines are typically derived from the reaction of fatty acids with polyamines that contain at least one tertiary amine group. Representative examples of amidoamines that can be used in hair care compositions include stearamidopropyl dimethyl amine and stearamidopropyl diethyl amine. One drawback of amidoamines is that their performance is often not as good as that of BTAC and CETAC.

There is still a need in the art for compounds or compositions that can deliver hair care performance that is better than CETAC and at least equal to if not better than BTAC, and that also have a better environmental profile than BTAC and CETAC.

### SUMMARY OF THE INVENTION

Surprisingly, it has now been found that compositions comprising one or more particular esteramines, either alone or in combination with mono- and diglycerides, can provide effective hair conditioning agents that deliver wet combing properties that are better than CETAC and at least equal to or better than BTAC. As used herein, “esteramine” is intended to encompass un-neutralized esteramine and esteramine in its neutralized, cationic salt form, unless the context clearly indicates otherwise. That esteramines can provide such improved properties is surprising because esteramines are utilized as intermediates to make esterquat actives, and are not typically known to be used as active components themselves.

One aspect of the present technology is directed to a composition that comprises (a) at least one esteramine, wherein the esteramine is the reaction product of a fatty acid source and an alkanolamine in a ratio of about 1.5 to 3 moles of acyl groups per mole of alkanolamine; and (b) an acid selected from the group consisting of lactic acid, citric acid, maleic acid, adipic acid, boric acid, glutamic acid, glycolic acid, formic acid, acetic acid, ascorbic acid, uric acid, oxalic acid, aspartic acid, butyric acid, lauric acid, glycine, ethane sulfonic acid, hydrochloric acid, sulfuric acid, phosphoric acid, and combinations thereof; wherein the acid is present in an amount sufficient to obtain a pH of about 2 to about 8 and neutralize the esteramine to form an esteramine salt, and wherein the esteramine is derived from a fatty acid source that has not been modified by self or cross metathesis.

In another aspect, the present technology is directed to a composition comprising:
(a) about 30% to about 99% by weight of the composition of one or more esteramines, and
(b) about 1% to about 70% by weight of a solvent,
   wherein the esteramine is derived from a fatty acid source that has not been modified by self or cross metathesis.

In a further aspect, the present technology is directed to a composition comprising (a) about 30% to about 100% by weight of the composition of a mixture of one or more esteramines and one or more glycerides, wherein the esteramines comprise about 50% to about 90% by weight of the mixture, and the glycerides comprise about 10% to about 50% by weight of the mixture; and (b) 0% to about 70% by weight of the composition of a solvent; wherein the esteramine is derived from a fatty acid source that has not been modified by self or cross metathesis.

In a still further aspect, the present technology is directed to a formulation comprising: (a) 0.01% to about 50% by weight of a composition active comprising at least one esteramine or a salt thereof, either alone or in admixture with at least one glyceride; (b) optionally, one or more additional components; and (c) diluent to balance the formulation to 100%. In one embodiment, the formulation is a hair conditioning composition.

In another aspect, the present technology is directed to a hair conditioning composition comprising: (a) 0.01% to about 50% by weight of one or more esteramine actives, wherein the esteramine actives are the reaction product of a fatty acid source and an alkanolamine in a ratio of about 1.5 to 3 moles of acyl groups per mole of alkanolamine; (b) optionally, one or more additional components; and (c) diluent to balance the formulation to 100%; wherein the hair care composition, when applied to a hair tress, provides a wet combing Dia-Stron maximum peak load of about 50 gram mass force (gmf) or less.

### BRIEF DESCRIPTION OF THE DRAWINGS

FIG. 1 is a graph comparing the wet combing results of hair conditioning compositions prepared with esteramine compositions of the present technology vs. compositions prepared with conventional quaternary ammonium compounds.

FIG. 2 is a graph comparing the wet combing results of hair conditioning compositions comprising palm fatty acid-based esteramines combined with different amounts of glycerides.

FIG. 3 is a graph comparing the wet combing results of hair conditioning compositions comprising either sunflower oil-based esteramines, sunflower fatty acid-based esteramines, or esteramine salts thereof.

FIG. 4 is a graph comparing the wet combing results of hair conditioning compositions comprising sunflower fatty acid-based esteramines, or esteramine salts thereof, combined with different amounts of glycerides.

FIG. 5 is a graph comparing the wet combing results of hair conditioning compositions comprising lauryl fatty acid-based esteramines, or esteramine salts thereof, combined with different amounts of glycerides.

FIG. 6 is a graph comparing the wet combing results of (1) hair conditioning compositions comprising either sunflower oil-based esteramines or palm oil-based esteramines; (2) hair conditioning compositions comprising either sunflower oil-based esteramine salts or palm oil-based esteramine salts; (3) hair conditioning compositions comprising either sunflower fatty acid-based esteramines, palm fatty acid-based esteramines, or lauryl fatty acid-based esteramines; and (4) hair conditioning compositions comprising either sunflower fatty acid-based esteramine salts, palm fatty acid-based esteramine salts, or lauryl fatty acid-based esteramine salts.

FIG. 7 is a graph demonstrating the synergistic effect that combining lauryl fatty acid-based esteramines, or esteramine salts thereof, with additional glycerides has on wet combing results, compared to lauryl fatty acid-based esteramines, or esteramine salts thereof, alone, and glycerides alone, in hair conditioning compositions.

FIG. 8 is a graph demonstrating the synergistic effect that combining palm fatty acid-based esteramines, or esteramine salts thereof, with additional glycerides has on wet combing results, compared to palm fatty acid-based esteramines, or esteramine salts thereof, alone, and glycerides alone, in hair conditioning compositions.

### DETAILED DESCRIPTION OF THE PREFERRED EMBODIMENTS

The compositions of the present technology comprise particular esteramines or salts thereof, which alone or in combination with glycerides, provide an effective hair conditioning agent that is biodegradable, and provides conditioning performance that is better than CETAC and comparable to or better than BTAC. The esteramines of the present technology also provide emulsification properties.

In general, the esteramines of the present technology are prepared by combining a natural oil or other fatty acid source and an alkanolamine, typically at a starting temperature at which the natural oil or fatty acid source is a liquid or molten, optionally adding a catalyst, then heating the reaction mixture until the desired composition, verified by acid value and alkalinity value, is reached. Reduced pressure may be applied during the reaction. The esteramines of the present technology can have a pH in the range of about 2 to about 9.5.

The fatty acid source for preparing the esteramines can be a variety of starting materials, such as free fatty acids, fatty acid esters, or acid chlorides corresponding to fatty acids. The fatty acid source is derived from a natural oil that has not been modified by self- or cross-metathesis. The free fatty acids can be separate, such as a single purified fatty acid, or in combinations, such as fatty acid mixtures characteristic of the fatty acid constituents of glyceride esters in natural oils. Fatty acid esters can be glycerides, such as mono-, di- and/or triglycerides, or alkyl esters of fatty acids, such as methyl esters or ethyl esters of fatty acids. The fatty acid esters can be derived from a single fatty acid, or mixtures of fatty acids, such as those derived from natural fatty acid feedstocks or from natural oils.

In some embodiments, the esteramines are prepared by the direct esterification of alkanolamines with the triglycerides in natural oils. When triglycerides are the source of the fatty acids, the resulting esteramine comprises a mixture of products that include diesteramines, monoesteramines, triglycerides, diglycerides, monoglycerides, glycerin, and free amine. Triglycerides may be obtained from various sources such as, but not limited to, sunflower oil, canola oil, soybean oil, palm oil, palm kernel oil, borage oil, pracaxi oil, walnut oil, jojoba oil, avocado oil, hempseed oil, rapeseed oil, and mixtures thereof. In some embodiments, it is desirable to use an oil having a large amount of unsaturation. Examples of such oils include, but are not limited to, sunflower oil, high oleic acid sunflower oil, canola oil, soybean oil, walnut oil, jojoba oil, borage oil, palm oil, and rapeseed oil, or mixtures thereof. Some preferred natural oils are those that comprise at least 30% by weight of polyunsaturated fatty acid groups. Examples of these oils include sunflower oil, comprising about 60% by weight linoleic acid, and borage oil, comprising about 40% by weight linoleic acid.

In other embodiments, the esteramines may be prepared from C8-32 fatty acids, or alkyl ester derivatives thereof, that are saturated, unsaturated, or a mixture of saturated and unsaturated fatty acids. The fatty acids may be derived from various natural oil sources such as, for example, sunflower, canola, corn, soybean, walnut, jojoba, palm, borage, and rapeseed, or mixtures thereof, that have not been modified by self or cross-metathesis. In some embodiments, the fatty acid can be a single, purified fatty acid, such as lauryl fatty acid, or a specific combination of fatty acids.

The alkanolamines useful for preparing the esteramines of the present technology correspond to the following general formula:

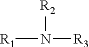

where R₁, R₂, and R₃ are independently selected from C₁₋₆ alkyl or hydroxy alkyl groups. Examples of alkanolamines include triethanol amine (TEA), methyl diethanolamine (MDEA), ethyl diethanolamine, dimethyl amino-N-(2,3-propanediol), diethylamino-N-(2,3-propanediol), methylamino-N,—N,-bis(2,3-propanediol), ethylamino-N,N-bis(2,3-propanediol), and mixtures thereof. In some embodiments, the alkanloamine comprises MDEA. In other embodiments, the alkanolamine comprises TEA. The molar ratio of triglyceride/fatty acid to alkanolamine is about 1.5 to 3 moles of acyl groups to 1 mole of amine.

In some embodiments, it may be desirable to neutralize the esteramine with an acid, forming an esteramine salt. The esteramine salt can be generated in-situ by reacting the corresponding esteramine with a sufficient amount of an acid to neutralize the esteramine to form a salt. The esteramine salt can have a pH in the range of about 2 to about 8, alternatively about 3 to about 7, alternatively about 3 to less than 7, alternatively about 3 to about 6, alternatively about 4 to about 6. In some embodiments, a stoichiometric amount of acid can be used for the neutralization. Alternatively, either an excess of acid or less than a stoichiometric amount of acid could be used, and less or more acid could then be added in the product formulation to adjust the pH of the final product to a desired level. Both organic and inorganic acids are suitable for in-situ reaction with an esteramine to generate the corresponding salts. Examples of acids include, but are not limited to, lactic acid, citric acid, maleic acid, adipic acid, boric acid, glutamic acid, glycolic acid, acetic acid, ascorbic acid, uric acid, oxalic acid, aspartic acid, butyric acid, lauric acid, glycine, formic acid, ethane sulfonic acid, hydrochloric acid, sulfuric acid, phosphoric acid, or combinations thereof. Preferred acids are those that do not contribute sulfates in order to prepare an esteramine salt that is sulfate-free. Hair conditioning compositions and other personal care compositions that are formulated from components that are sulfate-free are more desirable because they are milder and gentler on skin and hair than sulfate-containing compositions.

In addition to the esteramines, compositions of the present technology may comprise monoglycerides, diglycerides, triglycerides, or mixtures thereof. In some embodiments, the mono- and diglycerides are byproducts of the reaction between the alkanolamine and the natural oil triglycerides. In such embodiments, the fatty acid carboxylate groups in the mono- and diglycerides are derived from the natural oil source. It should be appreciated that small amounts of triglycerides, glycerol, and free amine may also be present. In other embodiments, glycerides are added as a separate component to the esteramines, and can have carbon chain lengths that are different from the fatty acid chain lengths in the esteramines. Regardless of the source, the mono- di- or triglycerides, or combinations thereof comprise saturated, unsaturated, or a mixture of unsaturated and saturated fatty acid carboxylate groups containing about 8 to about 32 carbon atoms. Typically, the glycerides have a ratio of mono- to diglyceride of about 1:1, although other ratios are also contemplated. When glycerides are included in the composition, the esteramine comprises about 50% to about 90%, alternatively about 50% to about 85%, alternatively about 60% to about 80%, alternatively about 65% to about 80%, alternatively about 70% to about 80% by weight, and the mono- and diglycerides comprise about 10% to about 50%, alternatively about 15% to about 50%, alternatively about 20% to about 40%, alternatively about 20% to about 35%, alternatively about 20% to about 30% by weight, based on the combined weight of the mixture of esteramine and glycerides.

Surprisingly, in some embodiments, the esteramines of the present technology, either alone or in admixture with glycerides, are in liquid form at room temperature (20° C.-25° C.) without the need for a solvent. Esteramines and esteramine salts prepared from sunflower oil, sunflower fatty acids, and lauryl fatty acids are all liquids at room temperature without solvents. Esteramines prepared from palm fatty acids are also in liquid form at room temperature, although esteramines prepared from palm oil, and the corresponding esteramine salts prepared from palm oil or palm fatty acids are solids at room temperature. The esteramines or esteramine salts in liquid form have low viscosities of about 3,000 cps or less, alternatively about 1,500 cps or less, alternatively about 1,000 cps or less, alternatively about 750 cps or less, alternatively about 600 cps or less, as measured at 20° C.-25° C. using a Brookfield viscometer, spindle LV2, with a spindle speed between 10 and 60 rpm depending on viscosity. In some embodiments, due to their low viscosity, mixtures of esteramines and glycerides can be free or substantially free of solvent, yet still be a liquid at room temperature. In such embodiments, the mixture of esteramines and glycerides comprises about 30% to about 100% by weight of the composition, with the esteramine comprising about 50% to about 90% by weight of the mixture, and the glycerides comprising about 10% to about 50% by weight of the mixture. The amount of solvent can be 0% up to about 70% by weight of the composition.

Advantageously, the low viscosity esteramine salts could be used to prepare highly concentrated product formulations, containing about 80% to about 99.9% by weight esteramine salt, and about 0.1 to about 3% by weight perfume, with other optional ingredients. These highly concentrated product formulations eliminate the need for water or other diluents or solvents, i.e., the amount of diluent or solvent can be 0%, and therefore can be packaged in smaller containers, providing an environmentally friendly formulation. It is envisioned that a consumer could dispense the product formulation from a small applicator, such as a dropper, onto the skin or hair, with the wash water serving to dilute the product for use.

If necessary or desired, the esteramines, or the mixture of esteramine and glycerides, can be diluted in particular solvents to form a liquid esteramine composition. In some embodiments, the solvents are those suitable for personal care. Examples of solvents include, but are not limited to, propylene glycol, glyceryl caprylate/caprate, glycerol monooleate, glycerin, sunflower oil, jojoba oil, alkyllactyl lactates, isopropyl alcohol, and combinations thereof. Glyceryl caprylate/caprate in liquid form is commercially available under the tradename STEPAN-MILD® GCC-L from Stepan Company, Northfield, Ill.

When used, the amount of solvent can range from about 1% to about 70%, alternatively about 5% to about 70%, alternatively about 10% to about 60%, alternatively about 10% to about 50%, alternatively about 10% to about 40%, alternatively about 10% to about 30% by weight, and the amount of esteramine or mixture of esteramine and glycerides can range from about 30% to 99%, alternatively about 30% to about 95%, alternatively about 40% to about 90%, alternatively about 50% to about 90%, alternatively about 60% to about 90%, alternatively about 70% to about 90% by weight of the composition. In some embodiments, the amount of solvent is about 1% to about 30% by weight, and the amount of the esteramine or mixture of esteramine and glycerides is about 70% to about 99% by weight, in order to form a high actives esteramine composition.

The esteramine compositions of the present technology have a variety of uses, and can be formulated into a variety of end use products. For example, the compositions can be used as a skin feel additive, a cationic emulsifier for skin care, a sun care additive, a textile treatment agent, or a leather conditioner. Examples of end use product formulations in which the esteramines can advantageously be used include, but are not limited to, hair conditioners, hair repair compositions, fabric softeners, fabric conditioners, hard surface cleaners, and skin care compositions. Product formulations can include the esteramine active component, either alone or in admixture with glycerides, in an amount of about 0.01% to about 50% by weight of the product formulation, alternatively about 0.05% to about 25%, alternatively about 0.1% to about 12%, alternatively about 0.01% to about 10%, alternatively about 0.1% to about 5%, alternatively about 0.5% to about 5%, alternatively about 1% to about 5%, alternatively about 2% to about 4% by weight of the product formulation.

The product formulations may contain other optional ingredients suitable for use, such as surfactants or other additives, and a diluent, such as water. Examples of surfactants include nonionic, cationic, and amphoteric surfactants, or combinations thereof. Examples of nonionic surfactants include, but are not limited to, fatty alcohol alkoxylates, polyalkylene glycols, mono- and/or dialkyl sulfosuccinates, fatty acid isethionates, fatty acid sarcosinates, fatty acid glutamates, ether carboxylic acids, alkyl oligoglucosides, and combinations thereof. Examples of cationics include, but are not limited to, BTAC, CETAC, and polyquaterniums, or combinations thereof. Examples of amphoteric surfactants include, but are not limited to, betaines, amidopropylbetaines, or combinations thereof. Other contemplated components include the long chain amido amines, such as stearamidopropyl dimethylamine (SAPDMA). Surfactant amounts in the product formulation can range from about 0.01% to about 10% by weight of the product formulation.

Examples of additives include rheological modifiers, emollients, skin conditioning agents, emulsifier/suspending agents, fragrances, colors, herbal extracts, vitamins, builders, enzymes, preservatives, antibacterial agents, or combinations thereof. For some product formulations, pH adjusters can be added to adjust the pH of the formulation to a pH in the range of about 1.5 to about 7.0, alternatively about 2.0 to about 6.5. Examples of pH adjusters that can be used include any of the acids mentioned above for neutralizing the esteramines. Total additives in the product formulation can range from about 0.01% to about 10% by weight of the product formulation.

Compositions of the present technology, comprising the esteramines or mixture of esteramines and glycerides, provide several benefits. The hair conditioning formulations comprising the compositions provide better wet hair combing properties compared to formulations comprising CETAC, and comparable or better wet hair combing properties compared to formulations comprising BTAC. However, unlike BTAC and CETAC, the esteramines of the present technology are readily biodegradable, and provide an improved environmental profile and lower toxicity compared to BTAC and CETAC. The compositions of the present technology provide improved wet combing properties without the need for additional components, such as fatty acid ethoxylates and alkyl and/or alkenyl oligoglucosides. Hair conditioning compositions comprising the esteramines or mixture of esteramines and glycerides of the present technology can be applied to the hair in an amount suitable for obtaining a hair conditioning effect. Suitable amounts of esteramine or esteramine and glyceride conditioning active applied to the hair can range from about 0.001% to about 5% by weight, alternatively about 0.001% to about 2%, alternatively about 0.002% to about 1.5%, alternatively about 0.025% to about 0.5%, alternatively about 0.025% to about 0.25% by weight, as measured on dry hair. The hair conditioning compositions provide a wet combing Dia-Stron Maximum Peak Load of about 50 gram mass force (gmf) or less, alternatively about 23 gmf or less, alternatively about 20 gmf or less, preferably about 8 to about 20 gmf, most preferably about 8 to about 15 gmf. In some embodiments, the esteramine actives provide a low viscosity liquid composition that is easily formulated with other ingredients, even at concentrations of up to about 50% by weight, to form a final product composition.

### EXAMPLES

The presently described technology and its advantages will be better understood by reference to the following examples. These examples are provided to describe specific embodiments of the present technology. By providing these examples, the inventors do not limit the scope and spirit of the present technology.

The following test methods are used to determine properties and performance of compositions of the present technology.

### Dia-Stron Procedure for Wet and Dry Combing

1. Rinse tress for 30 seconds.
2. Apply 0.5 mL of VO5® Volumizing Shampoo (non-conditioning shampoo).
3. Spread throughout tress.
4. Allow to air-dry.
5. Rinse tress for 30 seconds.
6. Apply 0.5 mL of Test Conditioner.
7. Spread throughout tress.
8. Affix tress to Dia-Stron MT1775 instrument and run “Wet Combing” procedure.
9. Repeat Step 8 nine more times.
10. Repeat Step 1-10 for 2 more tresses.
11. Allow tresses to air-dry.
12. Affix tress to Dia-Stron MT1775 instrument and run “Dry Combing” procedure.
13. Repeat Step 12 nine more times for one tress.
14. Repeat Steps 12-13 for 2 more tresses.

### Example 1: Preparation of Esteramine

### Esteramine Using Natural Oil

Natural oil in the desired amount was charged to a 3-liter, 4-necked glass reactor equipped with mechanical stirring, a reflux condenser, a thermocouple, and nitrogen blanketing. MDEA was added to the reactor in an amount to obtain a molar ratio of 2 fatty acid groups to 1 amine, and potassium carbonate was added to the reaction mixture. The reaction mixture was stirred at 300 rpm and heated to 160° C. under nitrogen until the free amine content stabilized between 2 and 3%. The resulting esteramine mixture was then cooled to room temperature. It should be appreciated that the resulting esteramine mixture comprises about 70% by weight esteramine, and about 30% by weight mono- and diglycerides as by-products from the reaction of MDEA and the natural oil.

### Esteramine Using Fatty Acids

Fatty acid in the desired amount was charged to a suitable sized reactor equipped with a mechanical stirrer, a thermocouple, and a simple distillation apparatus vented to a mineral oil-filled bubbler. A nitrogen sparging tube was attached to the remaining neck of the reactor, and the fatty acid was then sparged with nitrogen while stirring for no less than 1 hour. MDEA was added to the reactor in an amount to obtain a molar ratio of 1.7 moles of fatty acid per mole of amine. The reaction was slowly heated to 160° C. and held at this temperature until the acid value reached 0.06 meq/g or less. The resulting esteramine was then cooled to room temperature. It should be appreciated that when made with fatty acids rather than triglycerides in a natural oil, the resulting esteramine does not contain glycerides.

### Esteramine Salt

Esteramine in the desired amount was charged to a suitable sized reactor equipped with a magnetic stir bar. While stirring, lactic acid (85%) was added in portions until a stoichiometric amount of acid had been added. For the esteramine salts prepared with sunflower oil or sunflower fatty acids, the neutralization was performed at room temperature, since the sunflower derivatives were liquid at that temperature. The palm derivatives were low melting solids, and were heated to 35-45° C. until melted, and then the lactic acid was added. Once the addition was complete, the reaction mixture was allowed to stir until the mixture had reached room temperature. The resulting product is esterammonium lactate, an esteramine salt.

Sunflower oil-based, palm oil-based, and lauryl fatty acid-based esteramines and esteramine salts were prepared according to the above methods and had the following properties:

TABLE 1

| Esteramine | Viscosity (cps) | pH |
|---|---|---|
| Sunflower oil-MDEA | 66 | N/A |
| Sunflower FA-MDEA | 34 | N/A |
| Palm oil-MDEA | solid | N/A |
| Palm FA-MDEA | 38 | N/A |
| Lauryl FA-MDEA | 24 | N/A |
| Sunflower oil-MDEA salt | 553.5 | 5.74 |
| Sunflower FA-MDEA salt | 251 | 4.52 |
| Palm oil-MDEA salt | solid | 5.53 |
| Palm FA-MDEA salt | solid | 5.84 |
| Lauryl FA-MDEA salt | 233 | 5.63 |

The pH was measured on an Orion model 410A instrument in a 90:10 w/w solution of isopropyl alcohol (90%) and water (10%).

### Example 2: Preparation of Hair Conditioning Composition

Esteramines of the present technology and comparative hair conditioning components were formulated into hair conditioning compositions following the general procedure set forth below. Table 2 shows the general formula used to make the hair conditioning compositions.

TABLE 2

| | | % W/W in |
|---|---|---|
| Material Chemical Name | Function | Formulation |
| Deionized Water | Carrier | q.s. to 100.0′ |
| Natrosol ™ 250 HHR CS | Thickener | 0.7 |
| (Hydroxyethylcellulose) | | |
| Sodium Hydroxide | pH adjuster | q.s. |
| Esteramine or Cationic | Conditioning | Calculated for |
| | Active | 2% total solids |
| Cetyl Alcohol | Viscosity Modifier | 2.0 |
| Potassium Chloride, 10% | Opacifier | 0.5 |
| Solution | | |
| Citric Acid | pH adjuster | q.s. |
| Kathon ™ CG | Preservative | q.s. |
| (Methyl chloro | | |
| isothiazolinone/methyl | | |
| isothiazolinone mixture) | | |

### General Procedure

1. Charge water, begin mixing
2. Sprinkle in Natrosol 250 HHR CS
3. Adjust pH with 25% Sodium Hydroxide to target of pH 8-9. Mix until clear (30-40 min)
4. Heat to 70-75° C.
5. Add Conditioning component and mix until homogenous
6. Add Cetyl Alcohol and mix for 30 min.
7. Cool to 45° C. with mixing
8. In a small beaker dissolve Potassium Chloride in Water. Add to batch
9. Adjust pH 3.5-4 with 50% Citric Acid
10. Cool to Room Temp.
11. Add Kathon CG

Table 3 shows the different esteramines and comparative cationics used in the hair conditioning compositions prepared in accordance with the Table 2 formulation and the General Procedure. These hair conditioning formulations were used in the examples that follow.

TABLE 3
Standards

| | Maximum Peak | Std. |
|---|---|---|
| Molecule | Load (gmf) | Dev. |
| Shampoo | 243.6 | 28.1 |
| No Quat | 160.6 | 34.5 |
| BTAC | 19.9 | 4.0 |
| CETAC | 68.7 | 14.1 |
| GA-90 | 70.3 | 26.2 |

Palm Derivatives

| | Maximum Peak | Std. |
|---|---|---|
| Molecule | Load (gmf) | Dev. |
| 100% Palm Oil EA | 44.3 | 10.3 |
| 100% Palm Oil Salt | 52.3 | 13.4 |
| 100% Palm FA EA | 41.6 | 10.5 |
| 100% Palm FA Salt | 22.9 | 3.9 |

Palm Derivatives + GMO

| | Maximum Peak | Std. |
|---|---|---|
| Molecule | Load (gmf) | Dev. |
| 100% Palm FA EA | 41.6 | 10.5 |
| 90% Palm FA EA + 10% GMO | 44.5 | 13.9 |
| 80% Palm FA EA + 20% GMO | 23.2 | 6.4 |
| 70% Palm FA EA + 30% GMO | 10.6 | 2.1 |
| 60% Palm FA EA + 40% GMO | 33.7 | 7.0 |
| 50% Palm FA EA + 50% GMO | 53.2 | 8.6 |
| 100% Palm FA Salt | 22.9 | 3.9 |
| 90% Palm FA Salt + 10% GMO | 27.6 | 5.5 |
| 80% Palm FA Salt + 20% GMO | 13.5 | 2.1 |
| 70% Palm FA Salt + 30% GMO | 16.3 | 1.9 |
| 60% Palm FA Salt + 40% GMO | 35.9 | 6.8 |
| 50% Palm FA Salt + 50% GMO | 48.0 | 13.4 |

Sunflower Derivatives

| | Maximum Peak | Std. |
|---|---|---|
| Molecule | Load (gmf) | Dev. |
| 100% Sunflower Oil EA | 14.2 | 2.9 |
| 100% Sunflower Oil Salt | 14.7 | 1.8 |
| 100% Sunflower FA EA | 15.0 | 2.0 |
| 100% Sunflower FA Salt | 14.2 | 3.5 |

Sunflower Derivatives + GMO

| | Maximum Peak | Std. |
|---|---|---|
| Molecule | Load (gmf) | Dev. |
| 100% Sunflower FA EA | 15.0 | 2.0 |
| 90% Sunflower FA EA + GMO | 13.0 | 1.3 |
| 80% Sunflower FA EA + GMO | 14.7 | 2.2 |
| 70% Sunflower FA EA + GMO | 10.7 | 2.1 |
| 60% Sunflower FA EA + GMO | 20.1 | 4.5 |
| 50% Sunflower FA EA + GMO | 27.8 | 5.4 |
| 100% Sunflower FA Salt | 14.2 | 3.5 |
| 90% Sunflower FA Salt + GMO | 22.0 | 3.0 |
| 80% Sunflower FA Salt + GMO | 17.6 | 3.0 |
| 70% Sunflower FA Salt + GMO | 9.5 | 3.1 |
| 60% Sunflower FA Salt + GMO | 15.9 | 3.3 |
| 50% Sunflower FA Salt + GMO | 17.0 | 3.6 |

Lauryl Derivatives

| | Maximum Peak | Std. |
|---|---|---|
| Molecule | Load (gmf) | Dev. |
| 100% Lauryl FA EA | 87.1 | 15.8 |
| 100% Lauryl FA Salt | 54.2 | 10.8 |

Lauryl Derivatives + GMO

| | Maximum Peak | Std. |
|---|---|---|
| Molecule | Load (gmf) | Dev. |
| 100% Lauryl FA EA | 87.1 | 15.8 |
| 90% Lauryl FA EA + 10% GMO | 43.5 | 9.8 |
| 80% Lauryl FA EA + 20% GMO | 29.1 | 7.1 |
| 70% Lauryl FA EA + 30% GMO | 16.9 | 2.7 |
| 60% Lauryl FA EA+ 40% GMO | 18.7 | 4.2 |
| 50% Lauryl FA EA + 50% GMO | 36.3 | 12.0 |
| 100% Lauryl FA Salt | 43.5 | 9.8 |
| 90% Lauryl FA Salt + 10% GMO | 29.6 | 3.8 |
| 80% Lauryl FA Salt + 20% GMO | 17.7 | 4.8 |
| 70% Lauryl FA Salt + 30% GMO | 12.0 | 1.5 |
| 60% Lauryl FA Salt + 40% GMO | 18.0 | 4.3 |
| 50% Lauryl FA Salt + 50% GMO | 23.7 | 4.9 |

In Table 3, BTAC refers to behentrimonium chloride, CETAC refers to cetrimonium chloride (AMMONYX® CETAC-30 from Stepan Company, Northfield, Ill.), GA-90 refers to STEPANQUAT® GA-90, a palm and TEA-based esterquat from Stepan Company, Northfield, Ill.; GMO refers to DREWMULSE® GMO-K, a glycerol monooleate from Stepan Company, Northfield, Ill. It should be appreciated that the hair conditioning formulations have a pH of about 3.5-4.0, and that in this pH range, the esteramine is in a salt form. If the esteramine utilized to prepare the formulation is an esteramine salt, then the resulting formulation may contain a mixture of esteramine salts if the acid used to adjust the pH of the formulation is a different acid than that used to prepare the initial esteramine salt active.

### Example 3: Mechanical Wet Combing Comparative Evaluation

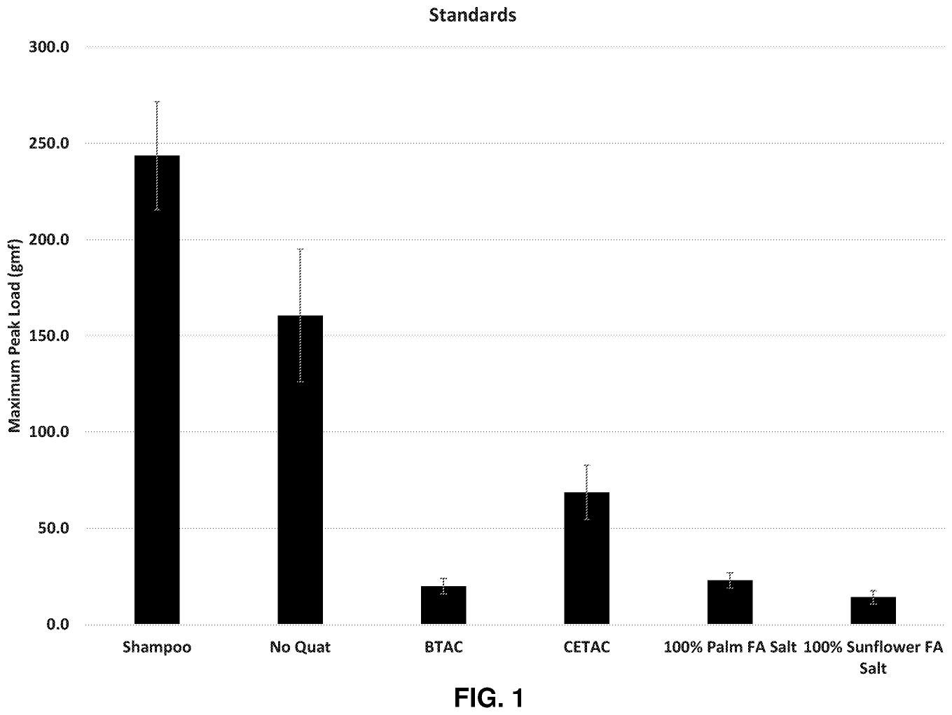

The inventive and comparative hair conditioning compositions in accordance with Example 2 were tested for wet combing ability using the Dia-Stron procedure previously described. Also tested were a conventional non-conditioning shampoo, and a blank conditioner formulation prepared in accordance with Table 2 except without an esteramine or cationic conditioning active. A comparison of the palm fatty acid esteramine salt and the sunflower fatty acid esteramine salt results with the CETAC, BTAC, shampoo, and blank conditioner standards results is shown in FIG. 1.

The FIG. 1 graph shows that the compositions prepared with the sunflower fatty acid esteramine salt had wet combing performance that was better than the CETAC and BTAC compositions. The results also show that compositions prepared with the palm fatty acid esteramine salt had better wet combing performance than the composition prepared with CETAC and fairly comparable performance to the composition prepared with BTAC.

### Example 4: Wet Combing Evaluation of Palm Oil-Based Esteramines with Varying Amounts of Glycerides

In this example, hair conditioning compositions were prepared to assess the effect of using different weight ratios of glycerides and palm oil-based esteramines, and salts thereof, on the wet combing properties of the hair conditioning compositions. Hair conditioning compositions were formulated using the Table 2 formulation, and using the following as the esteramine active in the different compositions:

Composition 1: 100% palm fatty acid esteramine

Composition 2: 100% palm fatty acid esteramine salt

Composition 3: 90% palm fatty acid esteramine and 10% glycerides

Composition 4: 90% palm fatty acid esteramine salt and 10% glycerides

Composition 5: 80% palm fatty acid esteramine and 20% glycerides

Composition 6: 80% palm fatty acid esteramine salt and 20% glycerides

Composition 7: 70% palm fatty acid esteramine and 30% glycerides

Composition 8: 70% palm fatty acid esteramine salt and 30% glycerides

Composition 9: 60% palm fatty acid esteramine and 40% glycerides

Composition 10: 60% palm fatty acid esteramine salt and 40% glycerides

Composition 11: 50% palm fatty acid esteramine and 50% glycerides

Composition 12: 50% palm fatty acid esteramine salt and 50% glycerides

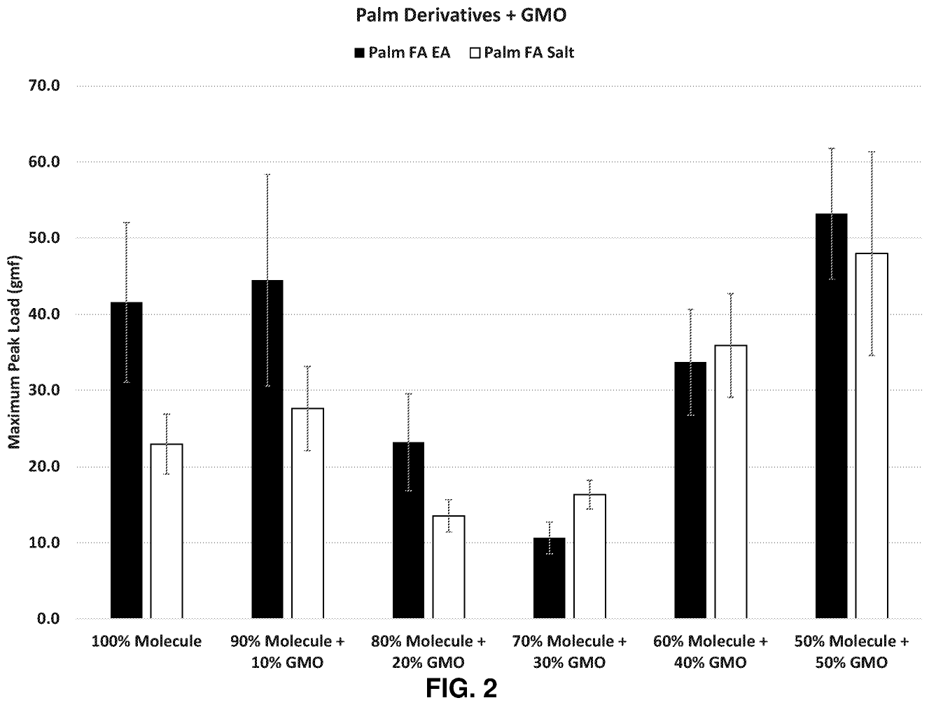

Each of the hair conditioning compositions was evaluated for wet combing ability using the Dia-Stron wet combing procedure. The results are shown in FIG. 2.

The graph in FIG. 2 shows that significant improvement in performance is achieved with a ratio of 70% esteramine and 30% glycerides, compared to esteramine alone (100% esteramine or esteramine salt) and compared to ratios of 90%/10%, 60%/40% and 50%/50% esteramine or esteramine salt and glycerides. Improved performance was also achieved with a ratio of 70% esteramine salt and 30% glycerides compared to esteramine or esteramine salt alone and compared to ratios of 90%/10%, 60%/40% and 50%/50% esteramine or esteramine salt and glycerides. Some improvement in performance was also achieved with a ratio of 80%/20%, particularly for the 80%/20% ratio of esteramine salt/glycerides.

### Example 5: Wet Combing Evaluation of Sunflower Oil-Based Esteramines

Sunflower oil-based esteramines were prepared from both sunflower oil and sunflower oil fatty acids, in accordance with the Example 1 procedure. Sunflower oil based esteramine lactate salts were also prepared in accordance with the procedure in Example 1. It should be understood that the esteramine and esteramine salt prepared with sunflower oil comprised about 70% by weight esteramine or esteramine salt and about 30% by weight glycerides coming from the oil. The esteramines and esteramine salts were formulated into hair conditioning compositions using the formulation in Table 2 and the General Procedure in Example 2. The viscosity of each of the hair conditioning compositions was measured at 25° C. using a Brookfield Digital Viscometer, LV 3 spindle, with a spindle speed of 12 rpm. The viscosity results are shown in Table 4:

TABLE 4

| Description of Active | Viscosity (cps) |
|---|---|
| Sunflower oil-MDEA | 6420 |
| Sunflower oil-MDEA salt | 11,400 |
| Sunflower FA-MDEA | 11,500 |
| Sunflower FA-MDEA salt | 5390 |
| Sunflower oil esterquat | 7200 |

The results in Table 4 show that the composition formulated with the sunflower oil esteramine salt, and the composition formulated with un-neutralized sunflower fatty acid esteramine had viscosities of 11,400 cps and 11,500 cps, respectively. This result is surprising because the actives themselves, namely the sunflower oil esteramine salt and the un-neutralized sunflower fatty acid esteramine, had low viscosities of 553.5 cps and 34 cps, respectively, at room temperature (See Table 1). Simply formulating these esteramines into a base hair conditioning formulation resulted in high viscosity products. Even more surprising is that the compositions comprising these esteramines had viscosities that were higher than the composition formulated with a sunflower oil-based esterquat. This is surprising because typically, esterquats are used for viscosity building, not esteramines.

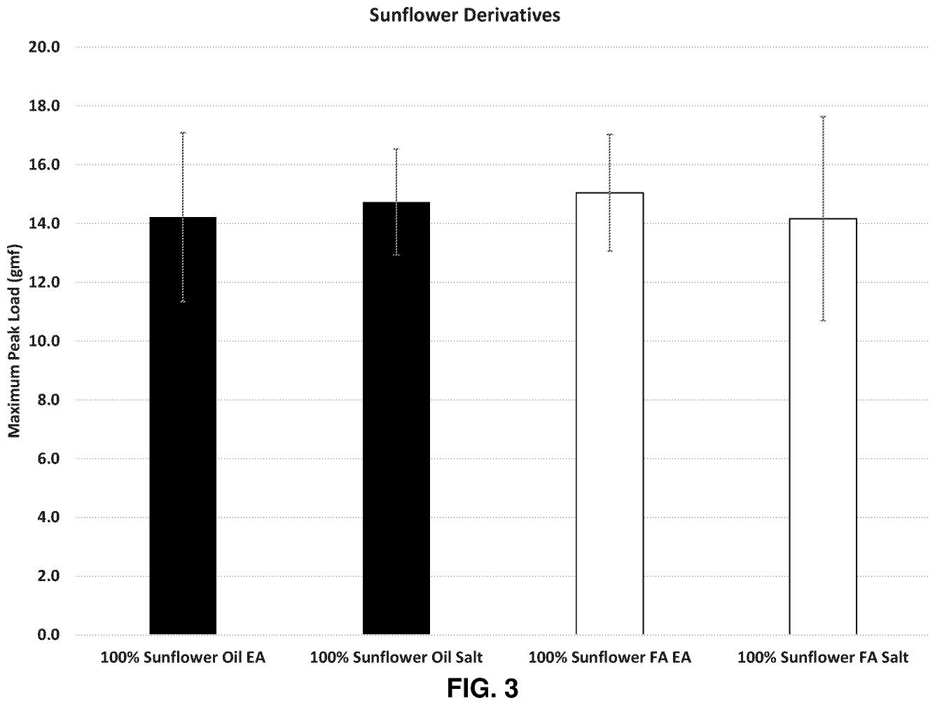

Each of the hair conditioning compositions was evaluated for wet combing ability using the Dia-Stron wet combing procedure, and the results are shown in FIG. 3. The graph in FIG. 3 shows that, surprisingly, all of the sunflower oil derivatives provided Dia-Stron Maximum Peak Loads in the range of 14-15 gmf. These results were achieved regardless of whether sunflower oil or sunflower oil fatty acids were used as the fatty acid source, and regardless of whether the esteramine or esteramine salt was used.

### Example 6: Wet Combing Evaluation of Sunflower Oil-Based Esteramines with Varying Amounts of Glycerides

In this example, hair conditioning compositions were prepared to assess the effect of using different weight ratios of glycerides and sunflower oil-based esteramines, and salts thereof, on the wet combing properties of the hair conditioning compositions. Hair conditioning compositions were formulated using the Table 2 formulation, and using the following as the esteramine active in the different compositions:

Composition 1: 100% sunflower fatty acid esteramine

Composition 2: 100% sunflower fatty acid esteramine salt

Composition 3: 90% sunflower fatty acid esteramine and 10% glycerides

Composition 4: 90% sunflower fatty acid esteramine salt and 10% glycerides

Composition 5: 80% sunflower fatty acid esteramine and 20% glycerides

Composition 6: 80% sunflower fatty acid esteramine salt and 20% glycerides

Composition 7: 70% sunflower fatty acid esteramine and 30% glycerides

Composition 8: 70% sunflower fatty acid esteramine salt and 30% glycerides

Composition 9: 60% sunflower fatty acid esteramine and 40% glycerides

Composition 10: 60% sunflower fatty acid esteramine salt and 40% glycerides

Composition 11: 50% sunflower fatty acid esteramine and 50% glycerides

Composition 12: 50% sunflower fatty acid esteramine salt and 50% glycerides

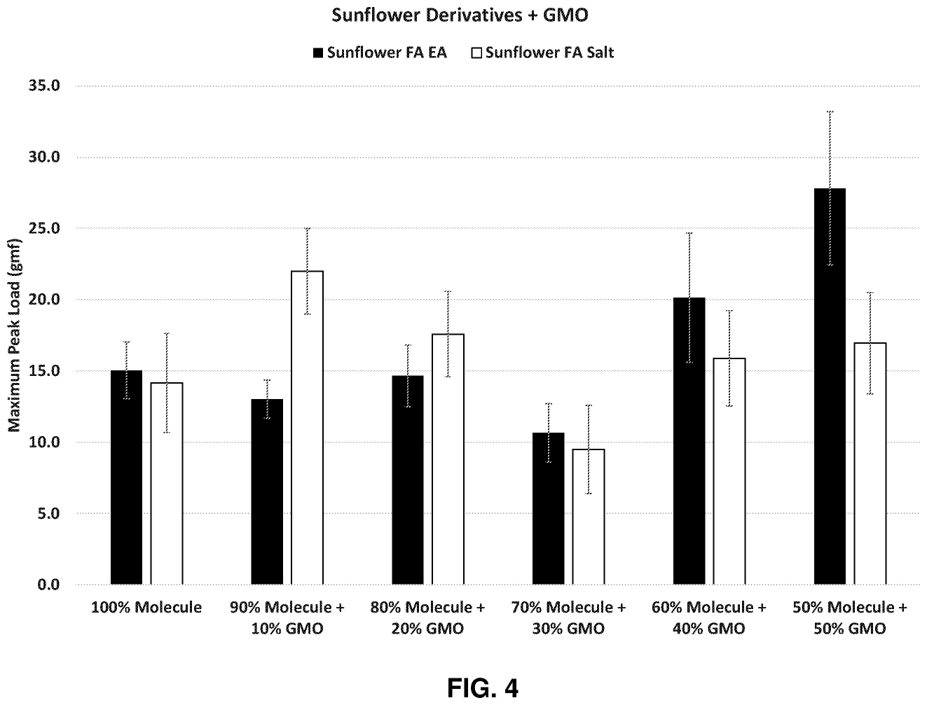

Each of the hair conditioning compositions was evaluated for wet combing ability using the Dia-Stron wet combing procedure. The results are shown in FIG. 4.

The graph in FIG. 4 shows that, even though the wet combing properties of 100% sunflower oil-based esteramines or esteramine salts are excellent (about 15 gmf Maximum Peak Load), improvement in performance is still achieved with a ratio of 70% esteramine or esteramine salt and 30% glycerides.

### Example 7: Wet Combing Evaluation of Lauryl Fatty Acid-Based Esteramines with Varying Amounts of Glycerides

In this example, hair conditioning compositions were prepared to assess the effect of using different weight ratios of glycerides and lauryl fatty acid esteramines, and salts thereof, on the wet combing properties of the hair conditioning compositions. Hair conditioning compositions were formulated using the Table 2 formulation, and using the following as the esteramine active in the different compositions:

Composition 1: 100% lauryl fatty acid esteramine

Composition 2: 100% lauryl fatty acid esteramine salt

Composition 3: 90% lauryl fatty acid esteramine and 10% glycerides

Composition 4: 90% lauryl fatty acid esteramine salt and 10% glycerides

Composition 5: 80% lauryl fatty acid esteramine and 20% glycerides

Composition 6: 80% lauryl fatty acid esteramine salt and 20% glycerides

Composition 7: 70% lauryl fatty acid esteramine and 30% glycerides

Composition 8: 70% lauryl fatty acid esteramine salt and 30% glycerides

Composition 9: 60% lauryl fatty acid esteramine and 40% glycerides

Composition 10: 60% lauryl fatty acid esteramine salt and 40% glycerides

Composition 11: 50% lauryl fatty acid esteramine and 50% glycerides

Composition 12: 50% lauryl fatty acid esteramine salt and 50% glycerides

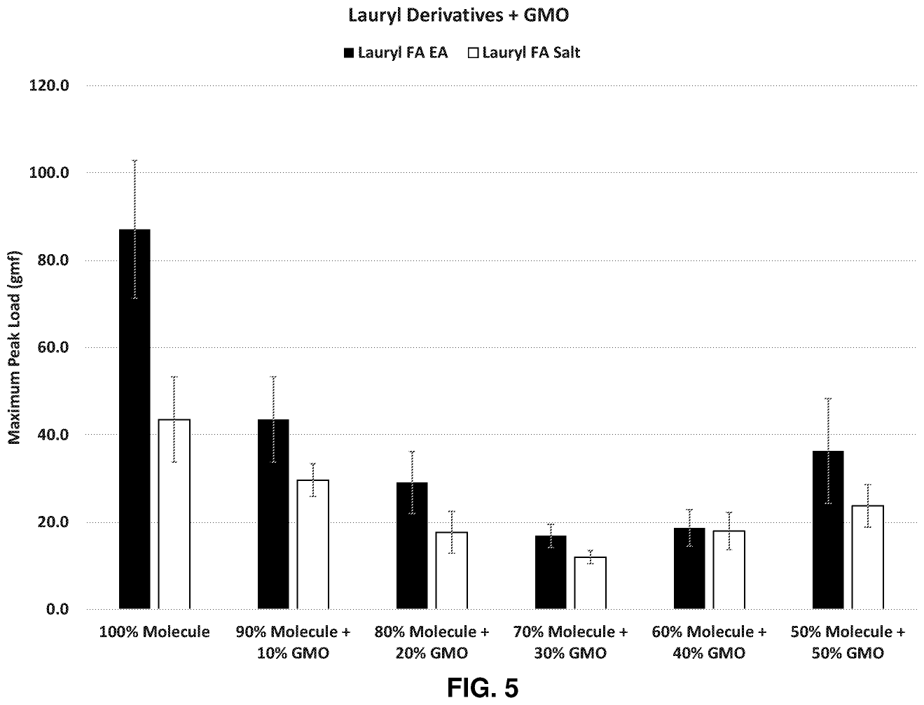

Each of the hair conditioning compositions was evaluated for wet combing ability using the Dia-Stron wet combing procedure, and the results are shown in FIG. 5.

The graph in FIG. 5 shows that significant improvement in performance was achieved with ratios of 80% esteramine or salt and 20% glycerides, 70% esteramine or salt and 30% glycerides, and 60% esteramine or salt and 40% glycerides, compared to esteramine alone (100% esteramine or esteramine salt). Some improved performance was also achieved with ratios of 90% esteramine salt and 10% glycerides, and 50% esteramine salt and 50% glycerides compared to esteramine or esteramine salt alone.

### Example 8: Wet Combing Evaluation of Esteramines from Different Oils

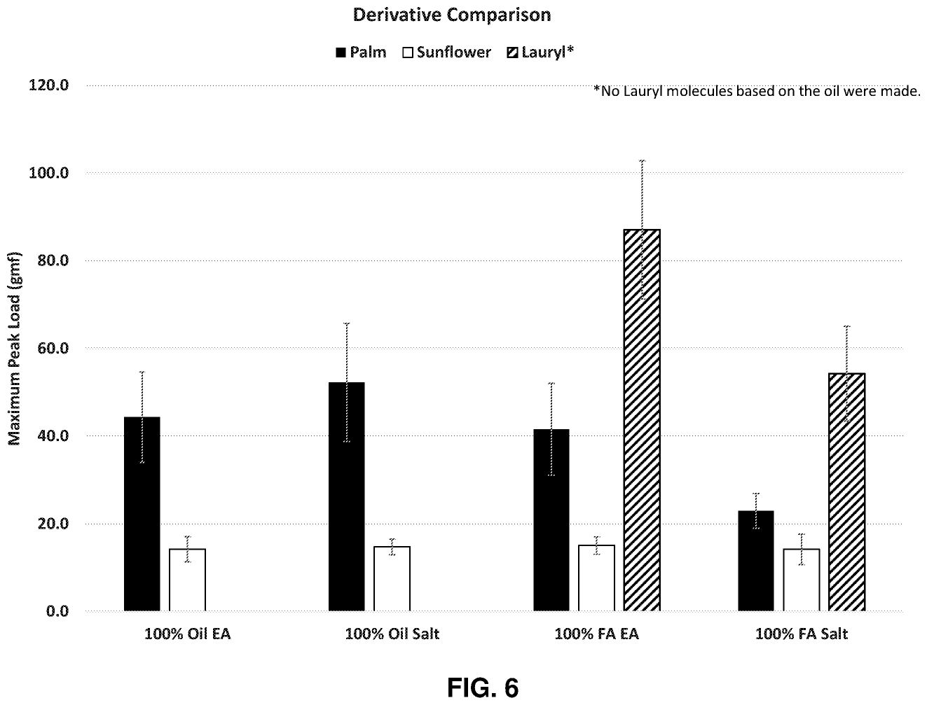

In this example, the hair conditioning compositions formulated from sunflower oil-based, palm oil-based, and lauryl fatty acid-based esteramines and salts were compared to evaluate the effect of using esteramines prepared from different oils or fatty acids on the wet combing properties of the hair conditioning compositions. The results are shown in FIG. 6.

The graph in FIG. 6 shows that better results were achieved with the esteramine/glycerides prepared from sunflower oil and sunflower fatty acids, indicating that carbon chain distribution in the starting oils or fatty acids has an effect on the performance of the resulting esteramine. Sunflower oil has appreciable amounts of linoleic acid (about 60%), which may indicate that the amount of polyunsaturation and/or the amount of unsaturated C18 in the starting oil has an effect on the performance of the resulting esteramine.

### Example 9: Wet Combing Evaluation Demonstrating Synergy

In this example, hair conditioning compositions were prepared to assess whether combining glycerides with a lauryl or palm fatty acid esteramine, or the esteramine salts thereof, can improve the wet combing properties of the hair care composition compared to compositions containing the fatty acid esteramine or esteramine salt alone, or the glyceride alone, as the conditioning agent. Hair conditioning compositions were formulated using the Table 2 formulation, and using 100% lauryl fatty acid esteramine, 100% lauryl fatty acid esteramine salt, 100% palm fatty acid esteramine, 100% palm fatty acid esteramine salt, 70% lauryl fatty acid esteramine/30% glycerides, 70% palm fatty acid esteramine/30% glycerides, or 100% glycerides as the conditioning active.

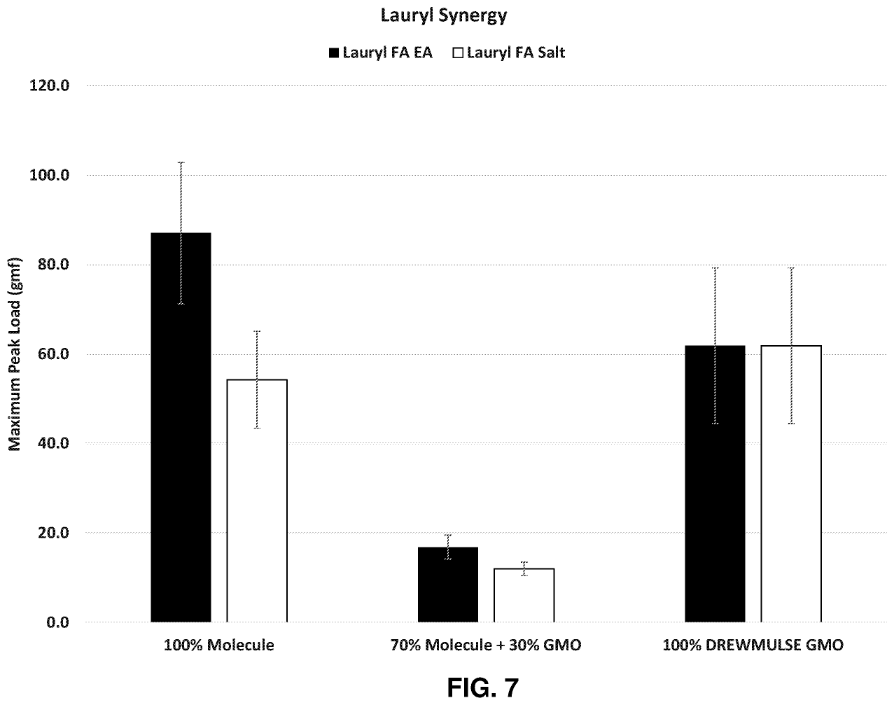

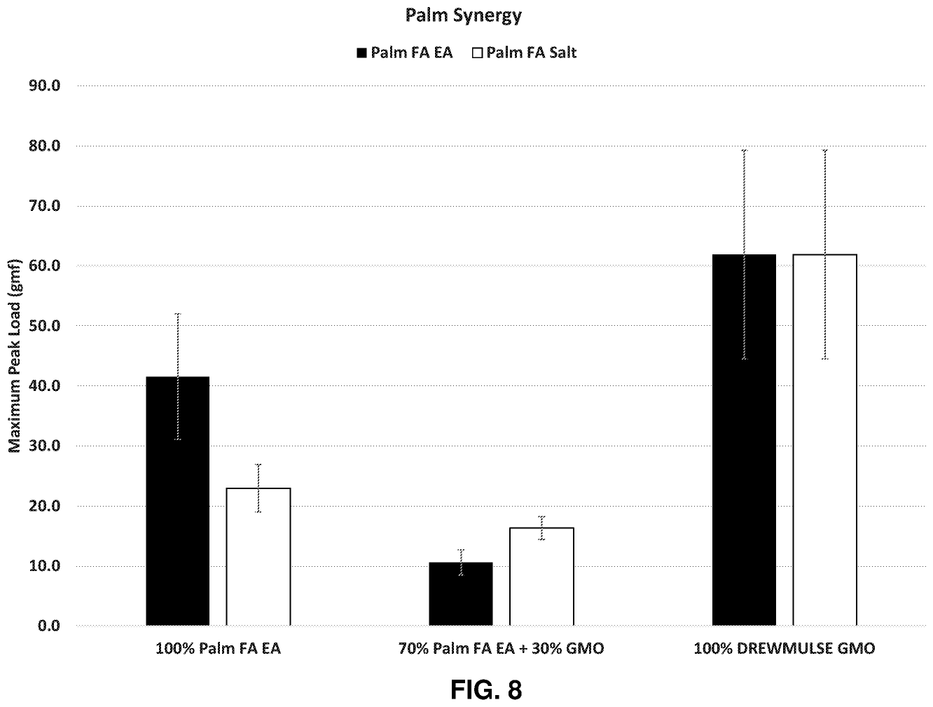

Each of the hair conditioning compositions was evaluated for wet combing ability using the Dia-Stron wet combing procedure. The results for the lauryl fatty acid derivatives are shown in FIG. 7, and the results for the palm fatty acid derivatives are shown in FIG. 8.

The graph in FIG. 7 shows that the compositions comprising the combination of 70% by weight lauryl fatty acid esteramine, or esteramine salt, and 30% by weight glycerides had significantly better wet combing properties compared to compositions comprising either the lauryl fatty acid esteramine alone, or the glycerides alone, as the conditioning active. The results in FIG. 7 demonstrate that the combination of lauryl fatty acid esteramine, or esteramine salt, and glycerides provides a synergistic mixture that imparts improved properties to a hair conditioning composition when compared to either the esteramine or glycerides alone.

The graph in FIG. 8 shows that the compositions comprising the combination of 70% by weight palm fatty acid esteramine, or esteramine salt, and 30% by weight glycerides had better wet combing properties compared to compositions comprising either the palm fatty acid esteramine alone, or the glycerides alone, as the conditioning active. The results in FIG. 8 demonstrate that the combination of palm fatty acid esteramine or esteramine salt and glycerides provides a synergistic mixture that imparts improved properties to a hair conditioning composition when compared to either the esteramine alone or glycerides alone, although the synergistic result is not as dramatic as that shown in FIG. 7 with respect to lauryl fatty acid.

The present technology is now described in such full, clear and concise terms as to enable a person skilled in the art to which it pertains, to practice the same. It is to be understood that the foregoing describes preferred embodiments of the present technology and that modifications may be made therein without departing from the spirit or scope of the present technology as set forth in the appended claims. Further, the examples are provided to not be exhaustive but illustrative of several embodiments that fall within the scope of the claims.

1. A composition comprising:
(a) about 30% to about 99% by weight of the composition of one or more esteramines, or salts thereof, and
(b) about 1% to about 70% by weight of a solvent, wherein the solvent is selected from the group consisting of propylene glycol, glyceryl caprylate/caprate, glycerol monooleate, glycerin, sunflower oil, jojoba oil, alkyllactyl lactates, isopropyl alcohol, and combinations thereof;
wherein the esteramine is derived from a fatty acid source that has not been modified by self or cross metathesis.

2. The composition of claim 1, wherein the esteramine is the reaction product of the fatty acid source reacted with an alkanolamine in a ratio of about 1.5 to 3 moles of acyl groups per mole of alkanolamine.

3. The composition of claim 2, wherein the fatty acid source is a natural oil.

4. The composition of claim 3, wherein the natural oil is selected from the group consisting of sunflower oil, canola oil, soybean oil, walnut oil, jojoba oil, palm oil, borage oil, rapeseed oil, and mixtures thereof.

5. The composition of claim 1, wherein the fatty acid source has a carbon chain length of 8 to 32 carbon atoms.

6. The composition of claim 2, wherein the alkanolamine is selected from the group consisting of triethanolamine (TEA), methyl diethanolamine (MDEA), ethyl diethanolamine, dimethyl amino-N-(2,3-propanediol), diethylamino-N-(2,3-propanediol), methylamino-N,—N,-bis(2,3-propanediol), ethylamino-N,N-bis(2,3-propanediol) and mixtures thereof.

7. The composition of claim 1, wherein the composition has a viscosity of about 3,000 cps or less at room temperature (20-25° C.).

8. The composition of claim 1, wherein the esteramine salt is selected from the group consisting of esterammonium lactate, esterammonium citrate, esterammonium glycolate, esterammonium formate, esterammonium aspartate, esterammonium chloride, esterammonium laurate, and combinations thereof.

9. The composition of claim 8, wherein the composition has a pH in the range of about 3 to about 6.

10. A composition comprising:
(a) about 30% to about 100% by weight of the composition of a mixture of one or more esteramines, or salts thereof, and one or more glycerides, wherein the esteramine or salt thereof comprises about 50% to about 90% by weight of the mixture, and the glycerides comprise about 10% to about 50% by weight of the mixture; and
(b) 0% to about 70% by weight of the composition of a solvent;
wherein the esteramine or salt thereof is derived from a fatty acid source that has not been modified by self or cross metathesis.

11. The composition of claim 10, wherein the glycerides comprise mono- and diglycerides.

12. The composition of claim 11, wherein the mono- and diglycerides have a carbon chain length of 8-32 carbon atoms.

13. The composition of claim 10, wherein the esteramine is the reaction product of a fatty acid source reacted with an alkanolamine in a ratio of about 1.5 to 3 moles of acyl groups per mole of alkanolamine.

14. The composition of claim 10, wherein the mixture is the direct esterification reaction product of sunflower oil reacted with methyl diethanolamine in a ratio of acyl groups to amine of 2:1.

15. The composition of claim 10, wherein the glycerides are separately added to the esteramine to obtain the mixture.

16. The composition of claim 10, wherein the solvent is selected from the group consisting of propylene glycol, glyceryl caprylate/caprate, glycerol monooleate, glycerin, sunflower oil, jojoba oil, alkyllactyl lactates, isopropyl alcohol, and combinations thereof.

17. A formulation comprising:
(a) 0.01% to about 50% by weight of a composition active, wherein the composition active comprises one or more esteramines that are the reaction product of a fatty acid source reacted with an alkanolamine in a ratio of about 1.5 to 3 moles of acyl groups per mole of alkanolamine;
(b) optionally, one or more additional components; and
(c) diluent to balance the formulation to 100%.

18. The formulation of claim 17, wherein the formulation is a hair conditioner, a hair repair composition, a fabric softener, a fabric conditioner, a hard surface cleaner, or a skin care composition.

19. The formulation of claim 17, wherein the formulation is a hair conditioning composition, and wherein the hair care conditioning composition, when applied to a hair tress, provides a wet combing Dia-Stron maximum peak load of about 50 gram mass force (gmf) or less.

20. The formulation of claim 17, wherein the esteramines are present in the formulation in an amount of 0.01% to about 12% by weight of the formulation.

21. The formulation of claim 17, wherein the composition active further comprises one or more glycerides, and the one or more glycerides and the one or more esteramines together comprise a mixture, wherein the one or more esteramines comprise about 50% to about 85% by weight of the mixture, and the one or more glycerides comprise about 15% to about 50% by weight of the mixture.

22. The formulation of claim 17, wherein the formulation further comprises a solvent, and the solvent is selected from the group consisting of propylene glycol, glyceryl caprylate/caprate, glycerol monooleate, glycerin, sunflower oil, jojoba oil, alkyllactyl lactates, isopropyl alcohol, and combinations thereof.

23. The formulation of claim 17, wherein the pH of the formulation is from about 1.5 to about 7.0.

24. The formulation of claim 19, wherein the maximum peak load is 23 gmf or less.
